(19) United States
(12) Patent Application Publication
(10) Pub. No.: US 2014/0346068 A1
(43) Pub. Date: Nov. 27, 2014

(54) MANAGEMENT TRAY UNIT

(71) Applicants: Yoshihito Omura, Tokyo (JP); Eiichiro Indei, Tokyo (JP)

(72) Inventors: Yoshihito Omura, Tokyo (JP); Eiichiro Indei, Tokyo (JP)

(73) Assignee: TOSHO INC., Tokyo (JP)

(21) Appl. No.: 14/344,745

(22) PCT Filed: Apr. 4, 2012
(86) PCT No.: PCT/JP2012/059206

§ 371 Date: Mar. 13, 2014

(30) Foreign Application Priority Data
Sep. 14, 2011 (JP) 2011-201094

(51) Int. Cl. A61J 1/00; A61B 19/02

(52) U.S. Cl. CPC A61J 1/00; A61B 19/02
USPC 206/363; 206/534; 206/538

(57) ABSTRACT

A management tray unit in which an image that allows reliable detection for each individual containing region may be obtained even with an imaging system and a degree of freedom of dividing a tray into individual containing regions is high. A composite partition member includes a base portion and a plurality of partition portions arranged in parallel with a predetermined interval. A connecting wall portion configured to connect one end of each of the partition portions is provided at the composite partition member. A space formed among the partition portion, the partition portion, and the connecting wall portion constitutes one individual containing region. A space formed by the partition portion and the connecting wall portion on the side of a second side of the base portion constitutes one individual containing region together with the partition portion of another composite partition member or a side wall of a tray.

### TECHNICAL FIELD

The present invention relates to a management tray unit for managing a use status of a medicine and/or a medical instrument to be used at a medical site. More specifically, the invention relates to a management tray unit configured to define a plurality of individual containing regions in a tray and individually contain a medicine and/or a medical instrument in each individual containing region, and used for grasping whether or not the medicine and/or the medical instrument has been used.

### BACKGROUND ART

Patent Documents 1 and 2 each disclose a management tray unit configured to contain a medicine and/or a medical instrument. These management tray units are each configured such that medicines and/or medical instruments are arranged on a plane (two-dimensionally) without overlapping with one another in an inner bottom of a tray and to individually manage each contained medicine and/or medical instrument. In the management tray unit of a first type disclosed in Patent Document 1, just an appropriate number of partition members with electronic tags attached thereto in advance are placed in the tray in appropriate disposition. A plurality of individual containing regions configured to individually contain the medicine and/or the medical instrument is thereby formed.

In the management tray unit of a second type disclosed in Patent Document 2, identification codes for the medicines and/or the medical instruments are printed on printing paper that can be laid on the inner bottom of the tray. A management system disclosed in Patent Document 2 includes disposition determination means for determining disposition of the medicines and/or the medical instruments to set in the tray according to a dispensing instruction, printing means for printing on the printing paper that can be laid on the inner bottom of the tray the identification codes for the medicines and/or the medical instruments corresponding to the disposition, imaging means for shooting the printing paper together with the tray from above, and determination means for reading the identification codes from an image obtained by the shooting and determining the presence or absence of use of each medicine and/or each medical instrument. The determination means determines that the medicine and/or the medical instrument in a corresponding location has been used, when the identification code can be read. The determination means determines that the medicine or the like in the corresponding location has not been used, when the identification code cannot be read. The determination means thereby determines the presence or absence of use of the medicine and/or the medical instrument based on a result of the reading.

In the management tray unit disclosed in Patent Document 2, the printing paper is laid on the inner bottom of the tray, and partition members of a removable type are held in the tray such that the partition members are superimposed on the printing paper. A large number of individual containing regions may be thereby readily formed inside the tray. Then, in this management tray unit, the identification code for the medicine and/or the medical instrument may be given to each individual containing region. Then, the tray is used for a medical activity such as an operation or a medical treatment, and the tray that has been returned from a medical site is placed below an imaging device, and is entrusted to the system. Then, the printing paper together with the tray are shot by the imaging means from the above, the identification code in each individual containing region is read from an image obtained by the shooting, and then presence or absence of the medicine and/or the medical instrument is determined based on a result of the reading. When performing image processing, an indirect method of reading the known identification code that appears or disappears according to the presence or absence of the medicine and/or the medical instrument in the individual containing region in this manner is used rather than a direct method of recognizing an image of a broad range of the medicines and/or the medical instruments. The presence or absence of use of the medicine and/or the medical instrument in each individual containing region is thereby accurately determined.

In the management tray unit disclosed in Patent Document 2, the partition members inserted into or removed from the tray are constituted from lattice-shaped members or members with portions of the lattice-shaped members cut off. These members are arranged at equal pitches or at unequal pitches. When the partition members are attached to the tray that is vacant by insertion, the internal space of the tray is partitioned into the large number of individual containing regions. In addition to the identification codes for the medicines and the medical instruments, related data such as the name of each set of the medicines and/or the medical instruments or the name of each medicine may be printed on the printing paper (refer to embodiment 1 of Patent Document 2, for example).

In the management tray unit used in the management system for medicines and/or medical instruments as mentioned above, a color bit code is introduced as the identification code. Generally, a bar code is printed on the surface of a sheet and is optically read. The color bit code represents encoded information using arrangement of colors. The color bit code (that may be also referred to as a 1D color bit code, a 1.5D color bit code, or a reflective color code) is developed (refer to Patent Documents 3 and 4, for example), as an identification code suitable for printing on or reading from a surface of each of various commodities. When reading the color bit code, image data including the color bit code is captured by the imaging device such as an area sensor and is divided into a plurality of color regions or a plurality of color region groups. Then, each divided color region or each divided color region group is narrowed down using a boundary condition or a number condition. Then, decoding is actually performed. When the color bit code is used, the decoding can be simply performed even if a cutout mark such as a two-dimensional bar code is not used.

### PRIOR ART DOCUMENTS

### Patent Documents

Patent Document 1: JP2009-125320A
Patent Document 2: JP2011-024663A
Patent Document 3: JP2008-287414A
Patent Document 4: JP2009-003721A

### SUMMARY OF THE INVENTION

### Technical Problem

In the management tray unit used in the management system for medicines and/or medical instruments with an imaging system disclosed in Patent Document 2, the identification codes for management of the medicines and/or the medical instruments are printed on the printing paper, and the printing paper is disposed between the tray and the partition members. Consequently, the printing paper without alteration may be a hindrance when the partition members are attached to the tray by insertion. The identification code may be therefore to be given to each individual containing region in a different way.

An object of the present invention is to implement a management tray unit in which an image that allows reliable detection for each individual containing region may be obtained even with an imaging system and a degree of freedom of dividing into a tray into individual containing regions is high.

### Solution to Problem

A management tray unit of the present invention comprises a tray configured to contain a medicine and/or a medical instrument and a plurality of partition members. The term “medicine” in the present invention includes a tablet, a powder medicine, and the like contained in a box, a bottle, and the like, and an auxiliary medicine to be used together with the medicine as well as an injection medicine such as an ampule, a vial, and a radiopaque dye. The term “medical instrument” includes various tools such as tweezers and scissors to be used for a medical treatment and an operation. The plurality of partition members are disposed inside the tray, positioned with respect to a bottom wall portion of the tray, and configured to define a plurality of individual containing regions for individually containing the medicine and/or the medical instrument inside the tray.

The plurality of partition members include a plurality of composite partition members each configured to define one or more of the individual containing regions independently or in cooperation with another partition member. The plurality of composite partition members each include: a base portion extending along the bottom wall portion and configured to be wholly or partially covered with the medicine or the medical instrument when the medicine or the medical instrument is contained in the individual containing region; and one or more partition portions extending from the base portion in a direction away from the bottom wall portion.

An identification code is provided on a surface of a section of the base portion of the composite partition member to be covered with the medicine or the medical instrument and is configured to identify the medicine or the medical instrument contained in the individual containing region defined by the composite partition member. The identification code is provided to be optically readable when the medicine or the medical instrument is not present on the section.

In the present invention, an attachment member is provided at each of the base portions of the composite partition members and the bottom wall portion of the tray. The attachment member forms an attachment configured to detachably attach the composite partition member to the bottom wall portion.

When an individual containing region is newly defined in the inside of the tray or one of the defined individual containing regions is changed in the management tray unit of the present invention as mentioned above, positions of one or more of the composite partition members are changed. The inside of the tray may be thereby divided into desired individual containing regions corresponding to medicines or the medical instruments to be contained. Further, by adopting the composite partition member having the identification code for the medicine or the medical instrument to be contained in a corresponding one of the individual containing regions one by one for each individual containing region, the identification code may be readily and accurately given to an inner bottom of each individual containing region. Collective imaging of the identification codes thus given to the respective individual containing regions is possible, as in the already-described management tray unit where the printing paper is placed between the tray and the partition members.

Then, when the management tray unit is used, the medicine or the medical instrument is set in each defined individual containing region of the tray, as in the already-described conventional medicine or medical instrument management system. The tray is then used for a medical activity such as an operation or a medical treatment. Then, the tray that has returned from a medical site is placed below an imaging device, and is entrusted to a system. Thereafter, the tray is automatically shot from above. Further, the identification code for machine reading in each individual containing region is read from an image obtained by the shooting. Use of the medicine or the medical instrument is detected, based on a result of the reading. In this case as well, an indirect method of reading the identification code that is known and appears or disappears according to presence or absence of the medicine or the medical instrument in each individual containing region is adopted, as in the already-described system. Use of the medicine and/or the medical instrument is accurately determined for each individual containing region.

Thus, according to the present invention, a management tray unit with a high degree of freedom of defining the individual containing regions may be implemented. In addition, in this management tray unit, an image that allows reliable detection for each individual containing region may be obtained even with an imaging system including a small number of sensors, which is easy to manufacture and maintain.

The attachment may be formed by a surface fastener comprising a fastener member including a hook surface and another fastener member including a loop surface. The attachment member provided at the bottom wall portion of the tray is one of the fastener member and the another fastener member of the surface fastener, and the attachment member provided at the base portion of the composite partition member is the other of the fastener member and the another fastener member of the surface faster. With this arrangement, the attachment member may be mounted conveniently and at low cost.

The attachment may have a structure configured to use permanent magnet magnetic poles. The attachment member provided at the bottom wall portion of the tray is one of the permanent magnet and a magnetic body to be attracted by the permanent magnet, and the attachment member provided at the base portion of the composite partition member is the other of the permanent magnet and the magnetic body to be attracted by the permanent magnet. With this arrangement, the attachment member may be mounted conveniently and at low cost. In addition, by placing the management tray unit in an appropriate magnetic field environment, an attractive force may be even temporarily adjusted.

The attachment member provided at the bottom wall portion of the tray may be formed of a plurality of positioning fitted portions arranged in a surface direction of the bottom wall portion of the tray at predetermined intervals, and the attachment member provided at the base portion of each composite partition member may be formed of one or more positioning fitting portions provided on a back surface of the base portion opposed to the bottom wall portion of the tray and configured to be fitted in one or more of the plurality of positioning fitted portions. With this arrangement, the inserting position of each composite partition member may be readily determined.

Preferably, the plurality of fitted portions are formed of a plurality of holes or concave portions formed in a plate disposed on the bottom wall portion of the tray in the form of a matrix, and the one or more fitting portions are formed of one or more convex portions configured to be fitted in the plurality of holes or concave portions. With this arrangement, the composite partition members may be quickly and orderly arranged.

Preferably, one or more of the plurality of composite partition members each include the base portion with a width longer than a unit length of pitch of the plurality of holes or concave portions, and a whole or a part of the identification code is attached to an upper surface section of the base portion distant from the partition portion by the unit length or more, whereby machine reading of the whole or the part of the identification code may not be performed when the whole or the part of the identification code is hidden. With this arrangement, the identification code in the individual containing region having a width of three times or more of the unit length of pitch is reliably hidden by the medicine or the medical instrument contained in the individual containing region. The identification code in the individual containing region having a width of twice or more of the unit length of pitch is also hidden by the medicine or the medical instrument contained in the individual containing region, with a high probability. Thus, reliability of detection based on collective imaging is increased.

The plurality of composite partition members may each include: the base portion having a rectangular contour; and a plurality of the partition portions extending along first and second sides of the base portion opposed to each other and arranged with predetermined intervals. A space formed between adjacent two of the plurality of the partition portions may constitute at least a section of the individual containing region. Such a composite partition member may independently form one or more of the individual containing regions without cooperating with a side wall of the tray or another partition member. Thus, the one or more individual containing regions may be more readily formed.

Preferably, when each composite partition member includes the plurality of partition portions as described above, the plurality of the partition portions are provided in parallel with the first and second sides, and a connecting wall portion configured to connect one end of each of the plurality of the partition portions is provided at the base portion along one of third and fourth sides opposed to each other and different from the first and second sides. With this arrangement, the medicine and/or the medical instrument is prevented from moving along the partition portion. Thus, there is no need for using another partition member or a side wall of the tray.

Each of the plurality of the partition portions adjacent to the first side may be provided to coincide with the first side, and each of the plurality of the partition portions adjacent to the second side may be provided apart from the second side. The composite partition member including such a plurality of partition portions includes the base portion having a width broader than the width of the individual containing region formed between two of the partition portions. Thus, even if the individual containing region is considerably thin or small, the medicine and/or the medical instrument may be stably fixed in the same position of the bottom wall portion of the tray. With this arrangement, even if a small object or a thin object is included in the medicine and/or the medical instrument to be handled, the individual containing region for stably containing any medicine and/or medical instrument is provided.

Further, each of the plurality of the partition portions adjacent to the first side may be provided apart from the first side, and each of the plurality of the partition portions adjacent to the second side may be provided apart from the second side. With this arrangement, the base portion has a broader width. Thus, the composite partition member is further stabilized.

The plurality of composite partition members may each include: the base portion having a rectangular contour; one of the partition portions extending along one of first and second sides of the base portion opposed to each other; and a connecting wall portion extending along one of third and fourth sides opposed to each other and different from the first and second sides and connected to one end of the partition portion. With this arrangement, the two sides of the base portion having no partition member extending therealong are opened. Thus, the medicine and/or the medical instrument that is large in size may be contained.

Preferably, the partition portion is provided at the base portion to coincide with the one of the first and second sides, and the connecting wall portion is provided at the base portion to coincide with one of the third and fourth sides. With this arrangement, the whole area of the base portion may be set to a whole or a section of one individual containing region. Thus, the medicine and/or the medical instrument that is larger in size may be contained.

It may be so arranged that the base portion becomes thinner as it is more distant from the partition portion, whereby an upper surface of the base portion is thereby inclined. With this arrangement, the medicine and/or the medical instrument may be held in a position distant from the partition portion.

In addition to the identification code, information capable of identifying the medicine or the medical instrument by mere visual observation may be displayed on an upper surface of the base portion. With this arrangement, the individual containing regions may be readily and accurately defined even if the defining is manually performed.

### BRIEF DESCRIPTION OF THE DRAWINGS

FIG. 1 shows a structure of a management tray unit or the like in a first embodiment of the present invention, and is an exploded perspective view of a tray and composite partition members.

FIG. 2A is a perspective view of the tray in which individual containing regions are defined.

FIG. 2B is a perspective view of the tray in which medicines and/or medical instruments are contained.

FIG. 3 is a perspective view of a cart.

FIG. 4A is a plan view of a composite partition member.

FIG. 4B is a perspective view of the composite partition member.

FIG. 4C is a front view of the composite partition member.

FIG. 4D is a right side view of the composite partition member.

FIG. 5A is a plan view of a composite partition member.

FIG. 5B is a perspective view of the composite partition member.

FIG. 5C is a front view of the composite partition member.

FIG. 5D is a right side view of the composite partition member.

FIG. 6A is a plan view of a composite partition member.

FIG. 6B is a perspective view of the composite partition member.

FIG. 6C is a front view of the composite partition member.

FIG. 6D is a right side view of the composite partition member.

FIG. 7A is a plan view of a composite partition member.

FIG. 7B is a perspective view of the composite partition member.

FIG. 7C is a front view of the composite partition member.

FIG. 7D is a right side view of the composite partition member.

FIG. 8A is a plan view of the composite partition member to which identification codes and medicine or medical instrument information display sections are attached.

FIG. 8B is a plan view of a variation example of the composite partition member to which the identification codes and the medicine or medical instrument information display sections are attached.

FIG. 9 is a plan view of a plurality of the composite partition members to which the identification codes and the medicine or medical instrument information display sections are attached.

FIG. 10 is a plan view of the composite partition member to which the identification code and the medicine or medical instrument information display section is attached.

FIG. 11 is a plan view showing a plurality of the composite partition members arranged on and attached to a bottom wall portion of the tray.

FIG. 12 is a perspective view of a checking unit for the tray that has been used.

FIG. 13 is a block diagram of a detection device.

FIG. 14 is a plan view of the tray in which the medicines and and/or the medical instruments are contained.

FIG. 15 shows a structure of a management tray unit or the like in a second embodiment of the present invention and is an exploded perspective view of a tray and composite partition members.

FIG. 16A is a perspective view of the tray in which individual containing regions are defined.

FIG. 16B is a perspective view of the tray in which medicines and/or medical instruments are contained.

FIGS. 17A to 17D are each a perspective view of a composite partition member.

FIGS. 18A to 18D are each a perspective view of a composite partition member.

FIG. 19 is a plan view of an intermediate layer member FIG. 20 is a plan view showing the intermediate layer member to which the partition members are attached.

FIG. 21 is a plan view of the management tray unit in which the medicines and/or medical instruments are further contained.

FIG. 22 is a sectional view of a portion of a tray and a composite partition member when a permanent magnet is used for an attachment member.

FIG. 23 is a sectional view of the composite partition member where an upper surface of a base portion is inclined.

FIG. 24 is a plan view of a composite partition member.

### DESCRIPTION OF EMBODIMENTS

Embodiments of a management tray unit that may be used in the management system disclosed in Patent Document 2 will be described below in detail with reference to the drawings. Illustration of a fastening tool such as a bolt, a connecting tool such as a hinge, and an electrical circuit, an electronic circuit, and the like is omitted in the appended drawings, for briefness or the like. The illustration is made, centering on components necessary for describing the invention and related to the invention.

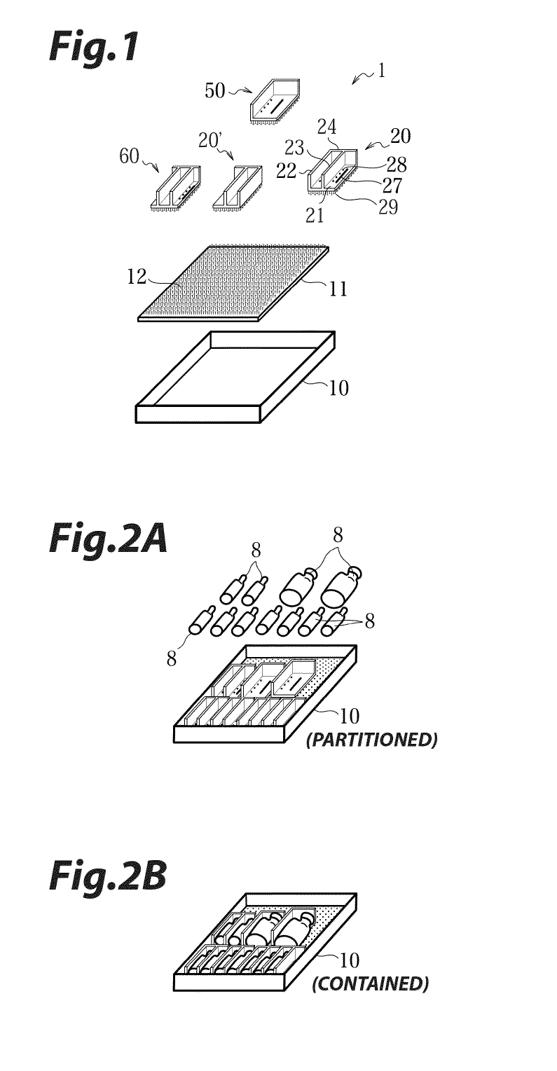

FIG. 1 is an exploded perspective view of a management tray unit 1 in a first embodiment of the present invention. The management tray unit 1 includes a tray 10 configured to contain a medicine and/or a medical instrument 8, a plate-like intermediate layer member 11 to be laid on an inner bottom of the tray 10, and a plurality of composite partition members 20, 20′, 50 and 60. FIG. 2A is a perspective view of the tray 10 and medicines and/or medical instruments 8 to be contained in this tray 10. FIG. 2A shows a state of the tray 10 in which the intermediate layer member 11 is disposed inside the tray 10 and the plurality of composite partition members 20, 50, and 60 are set on the intermediate layer member 11, thereby defining a plurality of individual containing regions inside the tray 10. FIG. 2B is a perspective view of the tray 10 in which the medicines and/or the medical instruments 8 are contained.

The tray 10 in this embodiment, which is vacant, has a shape of a box body like a shallow square plate. The tray 10 may have an arbitrary shape if the medicines 8 may be contained side by side, may be carried by hand and placed on a desk, and may be taken in and out from the plurality of individual containing regions defined by the composite partition members 20, 50, and 60.

The intermediate layer member 11 is configured to be snugly held in the tray 10 and then be fixed to the inner bottom of the tray 10. The intermediate member 11 may also be configured to be fixed to the inner bottom of the tray 10 with an appropriate hook or the like such that the intermediate layer member 11 does not move when inserted in the tray 10. The intermediate layer member 11 used in this embodiment has a structure in which one of a fastener member 12 including a hook surface and another fastener member 12 including a loop surface is attached to an upper surface of a plate member. The fastener member 12 including the hook surface and the another fastener member 12 including the loop surface comprise a surface fastener. The fastener member 12 in this embodiment is disposed on a whole area of the upper surface of the intermediate layer member 11 in the illustrated example. The fastener member 12, however, does not need to be provided for a region where the partition members 20, 50, and 60 are not attached.

The fastener member 12 is combined with each of fastener members 29, 59, and 69 respectively provided at the composite partition members 20, 50, and 60, thereby forming the surface fastener. It is enough to use a commercially available commodity for the surface fastener if the commodity functions as an attachment configured to detachably attach each base portion of the plurality of composite partition members 20, 50, and 60 to the bottom wall portion of the tray 10. To take an example, the surface fastener in which one of the fastener members 12 and 29 (59 or 69) includes the loop surface and the other of the fastener members 12 and 29(59 or 69) includes the hook surface is widely used, and is easy to use. When the fastener members 12 and 29(59 or 69) are made to face and contact each other, the loop surface and the hook surface are engaged with each other. A force for maintaining the contact state between the fastener member 12 and the fastener member 29(59 or 69) thereby functions, even if the fastener member 12 and the fastener member 29(59 or 69) are torn or laterally displaced.

The plurality of composite partition members 20, 50, and 60 are each attached to the bottom wall portion of the tray 10 through the intermediate layer member 11. The plurality of composite partition members 20, 50, and 60 partition the inside of the tray 10, thereby defining the plurality of individual containing regions. Each of the composite partition members 20, 50, and 60 used in this embodiment is not so large as to independently cover the whole area of the bottom wall portion of the tray 10. By combining a plurality of divided regions covered by the plurality or a large number of the composite partition members 20, 50, and 60, a necessary range of the bottom wall portion of the tray 10 is covered. The fastener members 29, 59, and 69 are attached to undersides of the respective composite partition members 20, 50, and 60. Each of these fastener members 29, 59, and 69 constitutes the surface fastener with the fastener member 12 of the intermediate layer member 11 on the bottom wall portion of the tray 10. As a result, the composite partition members 20, 50, and 60 are attached to the intermediate layer member 11 by bringing the fastener members 29, 59, and 69 into contact with the fastener member 12 of the intermediate layer member 11, and are fixedly held on the inner bottom of the tray 10 through the intermediate layer member 11. The composite partition members 20, 50, and 60 are fixed to the bottom wall portion of the tray 10 to partition the inside of the tray 10, thereby defining the plurality of individual containing regions associated with the composite partition members 20, 50, and 60.

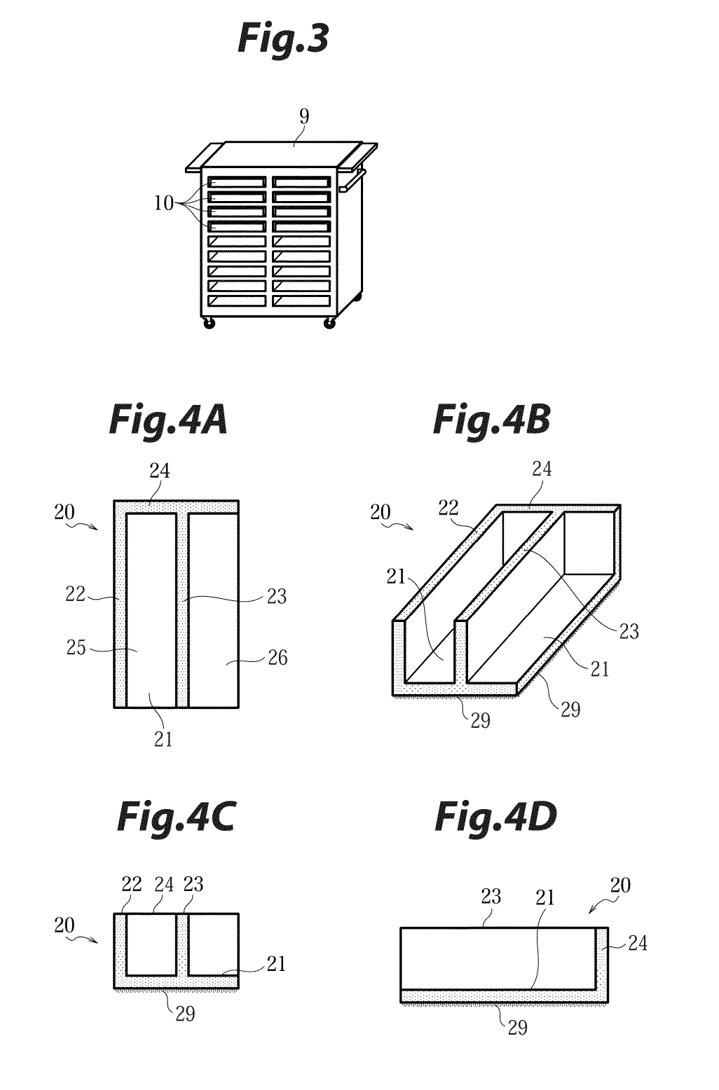

As shown in FIGS. 2A and 2B, the medicines and/or the medical instruments 8 are respectively contained in the individual containing regions corresponding to the plurality of the medicines and/or the medical instruments 8. Then, the tray 10 is received in a corresponding one of a plurality of shelves of a cart 9 as shown in FIG. 3.

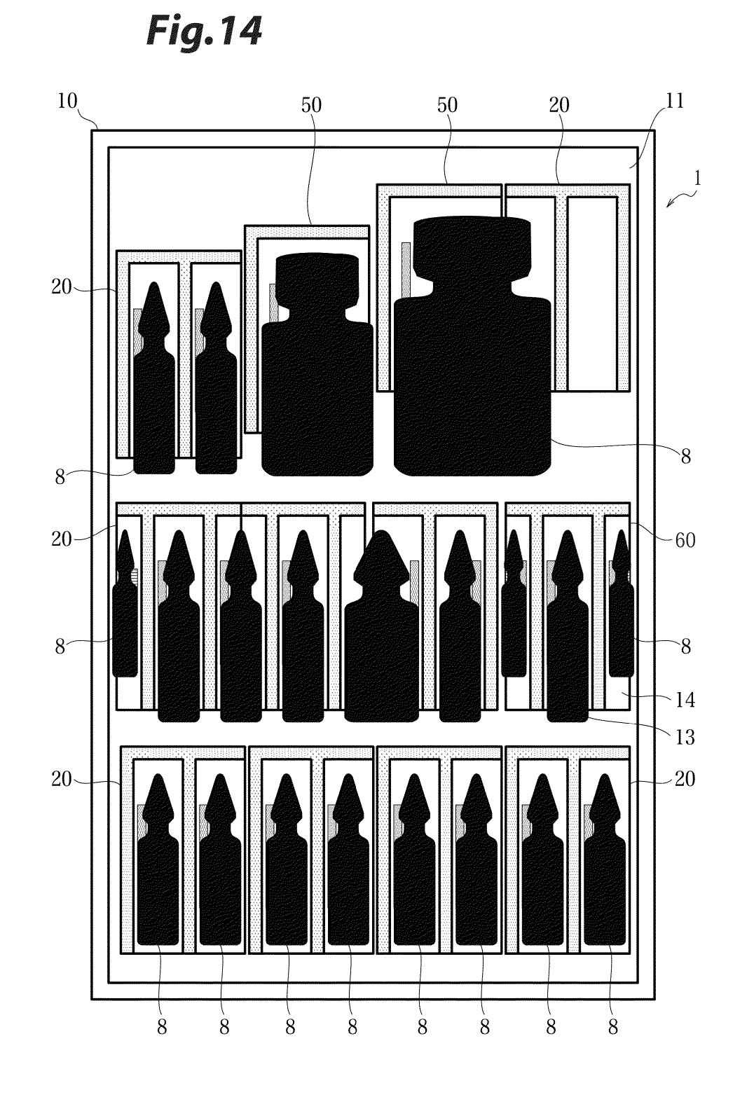

Three types of the composite partition members 20, 50, and 60 are used in this embodiment. As shown in FIGS. 4A to 4D, the composite partition member 20 includes a base portion 21 having a rectangular contour and a plurality of partition portions 22 and 23 extending along first and second sides of the base portion opposed to each other and arranged in parallel with a predetermined interval. The partition portion 22 adjacent to the first side is provided to coincide with the first side, while the partition portion 23 adjacent to the second side is provided apart from the second side. A connecting wall portion 24 is provided at the composite partition member 20. The connecting wall portion 24 extends along one of third and fourth sides opposed to each other and different from the first and second sides, and connects one end of each of the partition portions 22 and 23. A space 25 formed among the partition portion 22, the partition portion 23, and the connecting wall portion 24 constitutes one individual containing region. The fastener member 29 is provided at the bottom section of the base portion 21. A space 26 formed by the partition portion 23 and the connecting wall portion 24 on the side of the second side of the base portion 21 constitutes one individual containing region by using the partition portion of another composite partition member or a side wall of the tray 10 (refer to FIG. 14).

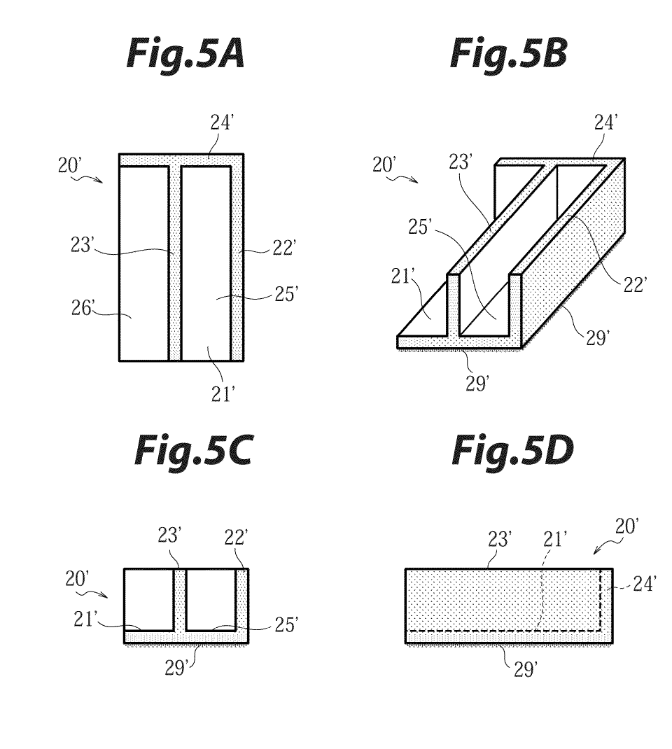

FIGS. 5A to 5D show a variation example of the composite partition member 20 shown in FIGS. 5A to 5D. By adding a mark “′” to reference numerals of components that are the same as the components of the composite partition member 20 shown in FIGS. 5A to 5D, description of this composite partition member 20′ is omitted. In this composite partition member 20′, a partition portion 22′ is located in a position reverse to the partition portion of the composite partition member 20.

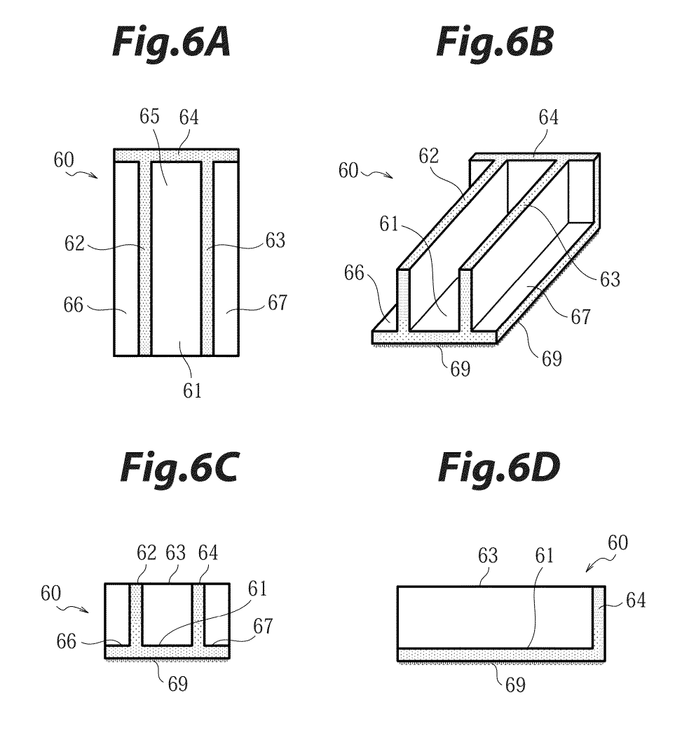

As shown in FIG. 6, the composite partition member 60 includes a base portion 61 having a rectangular contour and a plurality of partition portions 62 and 63 extending along first and second sides of the base portion 61 opposed to each other and arranged in parallel with a predetermined interval. The partition portion 62 adjacent to the first side is provided apart from the first side, and the partition portion 23 adjacent to the second side is also provided apart from the second side. A connecting wall portion 64 is provided at the composite partition member 60. The connecting wall portion 64 extends along one of third and fourth sides opposed to each other and different from the first and second sides, and connects one end of each of the partition portions 62 and 63. The fastener member 69 is provided at the bottom section of the base portion 61. A space 65 formed among the partition portion 62, the partition portion 63, and the connecting wall portion 64 constitutes one individual containing region. A space 66 formed by the partition member 62 and the connecting wall portion 64 on the side of the first side of the base portion 61 may individually constitute one individual containing region by using the partition portion of another composite partition member and a side wall of the tray 10, or may constitute a section of one individual containing region (refer to FIG. 14). Similarly, a space 67 formed by the partition portion 63 and the connecting wall portion 64 on the side of the second side of the base portion 61 may also individually constitute one individual containing region by using the partition portion of another composite partition member or a side wall of the tray 10, or may constitute a section of the one individual containing region.

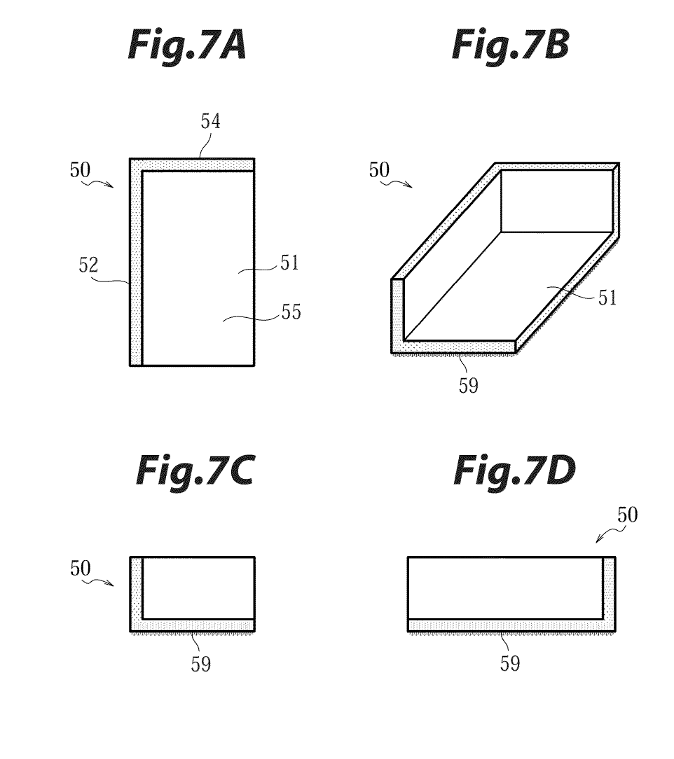

As shown in FIG. 7, the composite partition member 50 includes a base portion 51 having a rectangular contour and a partition portion 52 extending along a first side of the base portion 51 and provided to coincide with the first side. A connecting wall portion 54 is provided at the composite partition member 50. The connecting wall portion 54 extends along one of third and fourth sides not opposed to the first side and connected to one end of the partition portion 52. The fastener member 59 is provided at the bottom section of the base portion 51. A space 55 formed by the partition portion 52 and the connecting wall portion 54 may individually constitute one individual containing region or may constitute a section of one individual containing region by using the partition portion of another composite partition member or a side wall of the tray 10 (refer to FIG. 14).

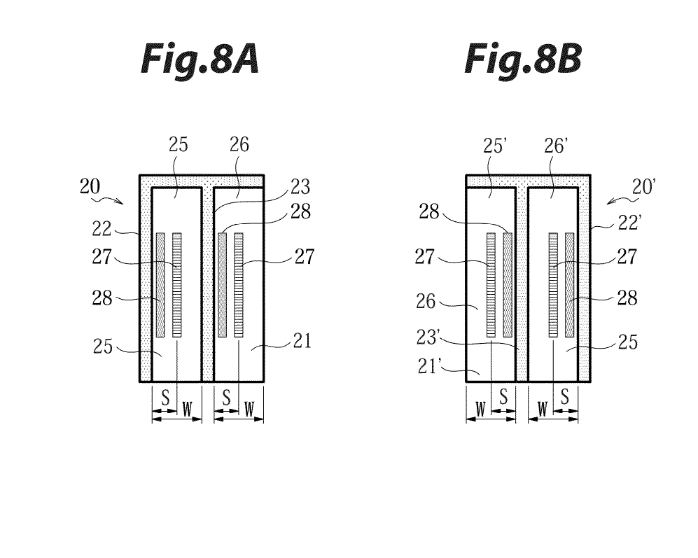

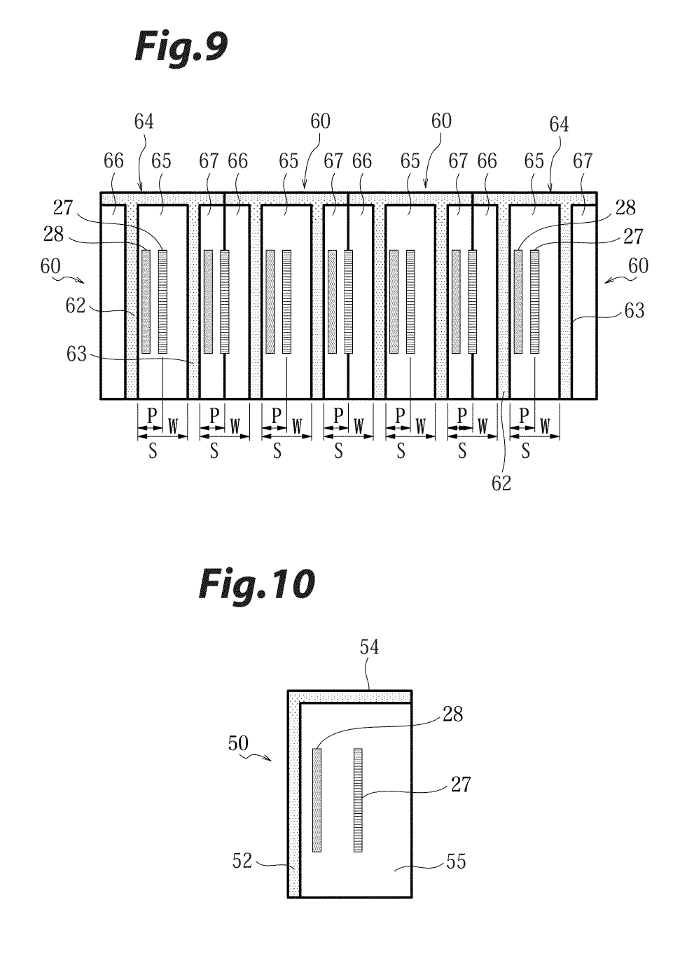

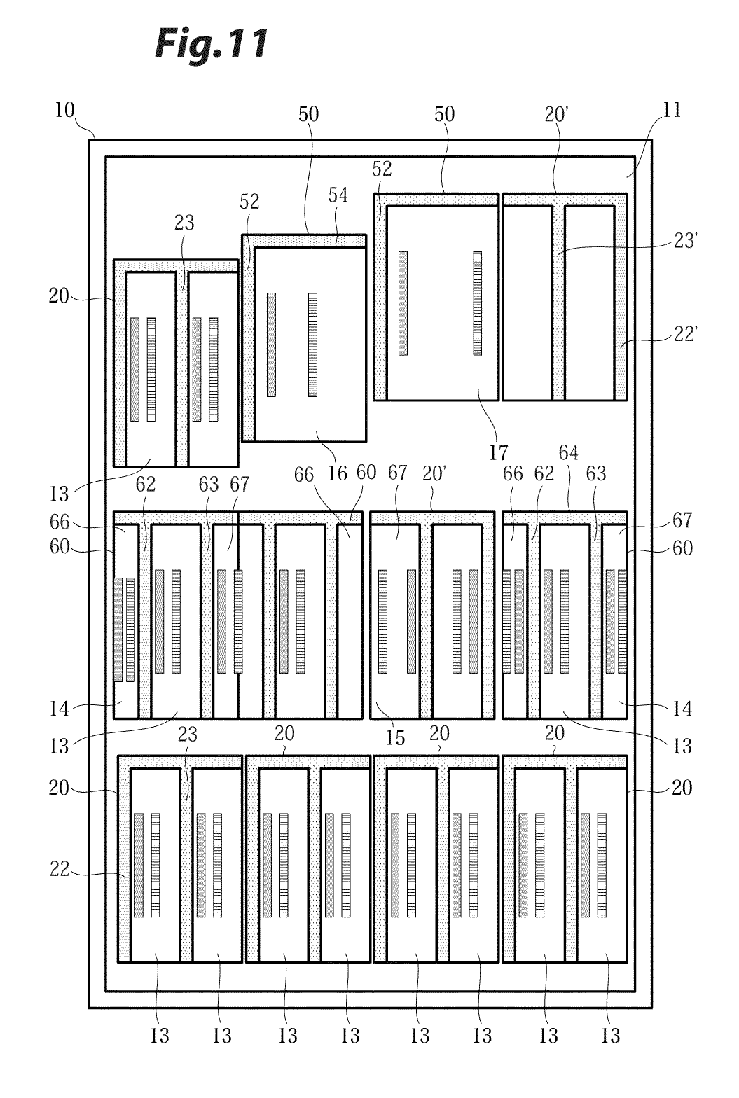

FIGS. 8A and 8B are plan views respectively showing a state where identification codes 27 and medicine or medical instrument information display sections 28 are attached to the composite partition member 20 and a state where the identification codes 27 and the medicine or medical instrument information display sections 28 are attached to the composite partition member 20′. FIG. 9 is a plan view showing a state where the identification codes 27 and the medicine or medical instrument information display sections 28 are attached to four composite partition members 60 arranged to define individual containing regions. FIG. 10 is a plan view showing a state where the identification code 27 and the medicine or medical instrument information display section 28 are attached to the composite partition member 50. FIG. 11 is a plan view of the management tray unit 1 in which the composite partition members 20, 20′ 50, and 60 are disposed inside the tray 10 to define a plurality of individual containing regions, and the medicine and/or the medical instrument 8 is contained in each of the plurality of individual containing regions. In these composite partition members 20, 20′ 50, and 60, the medicine or medical instrument information display sections 28 and the identification codes 27 for the medicine and/or the medical instruments 8 to be contained in the individual containing regions of the tray 10 associated with the composite partition members 20, 20′, 50, and 60 are directly printed on or attached to upper surfaces of the respective base portions 21, 21′ 51, and 61 of the composite partition members 20, 20′, 50, and 60 by using printing seals or the like. The composite partition members 20, 20′, 50, and each constitute code holding means for holding the identification code 27 on a bottom wall portion of the individual containing region. The identification code 27 is a mark for identifying the medicine and/or the medical instrument 8. A common medicine code used by a different dispensing machine or the like is usually used. Printing and display are specifically done using a 1D color bit code or a 1.5D color bit code. It is so arranged that, using a color printer, a linear display or a spiral display, for example, can be used. The medicine or medical instrument information display section 28 is a name for identifying the medicine and/or medical instrument 8. The medicine or medical instrument information display section 28 is printed and displayed with a character of gothic font capable of being identified just by visual observation.

The medicine or medical instrument information display section 28 described secondly is attached to an upper surface section of each of the base portions 21, 21′, 51, and 61 of the composite partition members 20, 20′, 50, and 60 close to one of the partition portions 22, 23, 22′, 23′, 52, 62, and 63. On contrast therewith, the identification code 27 described firstly is attached to an upper surface section of each of the base portions 21, 21′, 51, and 61 that are distant from the partition portions 22, 23, 22′, 23′, 52, 62, and 63. The widths of the spaces 25 and 65 and a space 25′ each completely match a width W of the individual containing region associated with a corresponding one of the spaces 25, 25′, and 65. Thus, a horizontal location S of the identification code 27 from the partition portion is set to the position that is approximately a half of the width W of the corresponding individual containing region (refer to FIGS. 8 and 9).

Also when the width of each of the space 26 and a space 26′ completely or generally matches the width W of the individual containing region associated with a corresponding one of the spaces 26 and 26′ (refer to FIG. 8), and also when a width obtained by combining the widths of the spaces 66 and 67 completely or generally matches the width of the individual containing region associated with the spaces 66 and 67 (refer to FIG. 9), the horizontal location S of the identification code 27 is set to the position that is approximately the half of the width W of the corresponding individual containing region.

The horizontal location S of the identification code 27 as mentioned above is a position where a whole or a portion of the identification code 27 is hidden by the medicine and/or the medical instrument 8, whereby machine reading of the identification code 27 cannot be performed from the corresponding individual containing region when the medicine and/or the medical instrument 8 is contained in the corresponding individual containing region.

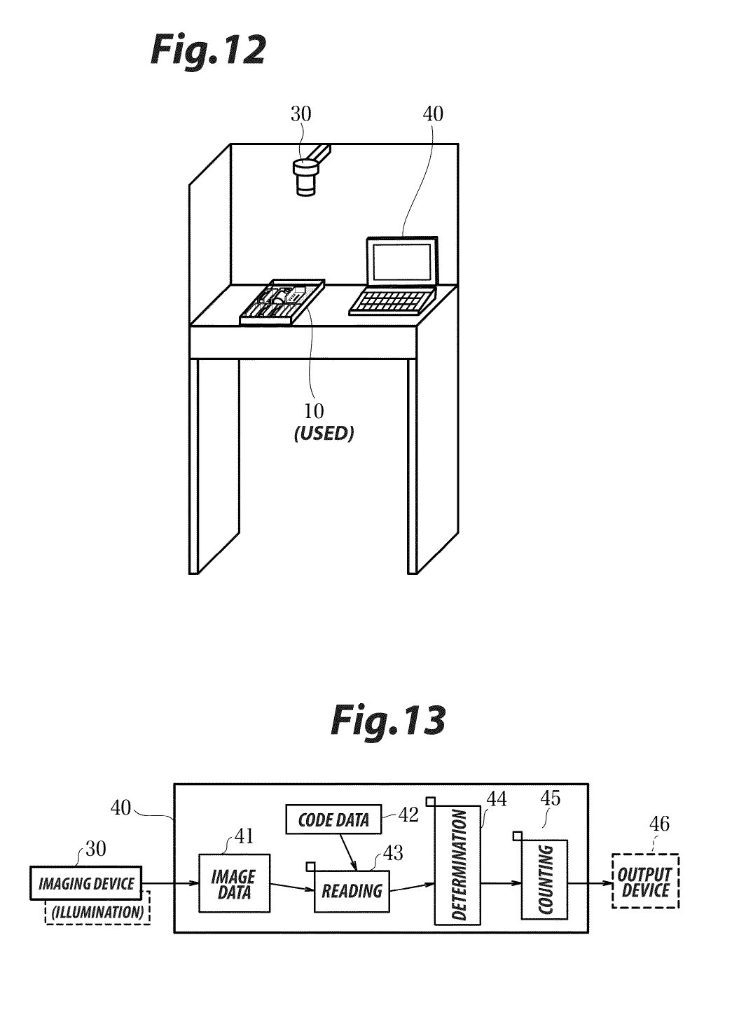

FIG. 12 is a perspective view of a working desk including the tray 10 that has been used and a checking unit (constituted from an imaging device 30 and a detection device 40 comprising a computer, or the like) configured to detect the medicine and/or the medical instrument 8 taken from the used tray 10. FIG. 13 is a block diagram showing various types of means inside the detection device 40. The various means comprise a computer program. A medicine management system using this management tray unit 1 includes the management tray unit 1, the imaging device 30 as imaging means for shooting the tray 10 from above, and the detection device 40 as detection means for detecting use of the medicine and/or the medical instrument 8 by reading the identification code 27 from an image obtained by the shooting by the imaging device 30.

The imaging device 30 and the detection device 40 are mounted on the working desk to constitute the checking unit.

A CCD camera, for example, is adopted as the imaging device 30 (refer to FIGS. 12 and 13), and is installed above an upper surface of the desk with an imaging direction facing downward. The imaging device 30 operates when the tray 10 is placed in a shooting range on the desk, and then shoots the tray 10 from the above. In this embodiment, the identification code 27 given to the upper surface of each of the base portions 21, 21′, 51, and 61 of the composite partition members 20, 20′, 50, and 60 attached to the bottom wall portion of the tray 10 is printed in color. Thus, the imaging device capable of color shooting is adopted such that an image to be shot by the imaging device may also be accurately obtained.

The detection device 40 comprises a so-called programmable information processing device such as a laptop computer, receives image data from the imaging device 30, and then outputs to an output device 46 a result of counting for a use status of the medicines and/or the medical instruments 8, or the like. In order to achieve that function, data regions of image data 41 and code data 42 are assigned to a memory in the information processing device comprising the detection device 40, and a reading program 43, a determination program 44, and a counting program 45 are installed in the information processing device.

The image data 41 is a color image obtained by shooting the surface of the medicines set in the tray 10 where the composite partition members 20, 20′, 50, and 60 with the identification codes 27 given thereto are attached to the bottom wall portion of the tray 10. The shooting is performed by the imaging device 30. This detection device 40 is configured to use an image obtained by shooting the used tray 10, in particular, as the image data 41.

The code data 42 is pattern information necessary for reading each identification code 27. Data configured to define the 1D color bit code or the 1.5 D color bit code is registered in advance.

The reading program 43 is configured to try code cutting and code recognition for the image data 41 each time the image data 41 is obtained and to read the identification code 27 from the image of the image data, using the code data 42 (refer to Patent Documents 3 and 4, for example).

The determination program 44 is configured to detect use of the medicine and/or the medical instrument 8, based on a result of reading the identification code by the reading program 43. The determination program 44 is configured to determine that the medicine and/or the medical instrument 8 associated with the identification code 27 has been taken out of the tray 10 already used and used when the reading program 44 can read the identification code 27.

The counting program 45 is configured to count a used quantity of the medicines and/or the medical instruments for each type of the medicines and each type of the medical instruments based on a result of determination by the determination program 44 and to count the medicines and/or the medical instruments for each tray or for each date on an appropriate basis to output results of the counting to the output device 46.

It is enough to use, for the output device 46, a device capable of outputting a result of use of one or more of the medicines and/or the medical instruments in a visually recognizable state or a state that allows data management or the like. The output device 46 may be a display attached to the detection device 40, a printing device such as a printer, or a communication unit configured to transmit electronic data to an inventory management system, a picking system, or the like.

A use mode and an operation of the medicine management system using the management tray unit 1 in this embodiment will be described by citing the drawings. This medicine management system is basically placed at a medicine dispensing location such as an in-house pharmacy or a nurse center. An operation of placing the medicine and/or the medical instrument 8 in the tray 10 and a process of detecting the medicine and/or the medical instrument 8 taken out from the tray 10 by the checking unit (30, 40) are also performed at the medical dispensing location. The medicine and/or the medical instrument 8 is, however, usually taken out from the tray 10 and is used at a different location such as an operation room.

Details of the operation and the process will be described below according to an operation procedure, in the order of defining individual containing regions inside the tray 10 that is vacant, placing the medicine and/or the medical instrument 8 in each of the individual containing regions of the tray 10 that has been partitioned, taking out the tray 10 containing the medicines and/or the medical instruments, bringing in the tray 10 used, and detecting the medicine and/or the medical instrument 8 used.

First, the intermediate layer member 11 is laid on the inner bottom of the vacant tray 10, in order to define the individual containing regions in the vacant tray 10 (refer to FIG. 1). Then, operations of attaching the composite partition members 20, 20′, 50, and 60 are appropriately repeated, thereby forming the partitioned tray 10 (refer to FIGS. 2 and 13). Then, the inside of the partitioned tray 10 is partitioned by the partition portions 22 and 23 of the composite partition member 20, the partition portions 22′ and 23′ of the composite partition member 20′, the partition portion 52 of the composite partition member 50, the partition portions 62 and 63 of the composite partition member 60 and the connecting wall portions 24, 24′, 54, and 64 to be divided into the individual containing regions that are large in number. Each individual containing region has a shape close to a rectangle as viewed in planar view. Formation of some of the individual containing regions of a same size or different sizes, determination of disposition of the individual containing regions, and a change in the disposition of the individual containing regions may be readily and quickly performed by attachment or removal of each of the composite partition members 20, 20′ 50, and 60.

To take an example, slightly narrow individual containing regions 13 of a width between the partition portions 22 and 23, between the partition portions 22′ and 23′, and between the partition portions 62 and 63, individual containing regions 14 of a minimum width defined by one of the spaces 66 and 67 of the composite partition member 60, individual containing regions 15 that are defined by a combination of the spaces 66 and 67 of the composite partition member 60 and are slightly broader than the slightly narrow individual containing regions 13, slightly broad individual containing regions 16 and 17 each defined by the composite partition member 50, and the like may be readily formed (refer to FIG. 11). The individual containing region of each of the above-mentioned sizes is formed in the following manner so as to be associated with the medicine and/or the medical instrument 8 to be contained, in one-to-one correspondence. That is, the individual containing region is formed such that a set of the identification code 27 and the medicine or medical instrument information display section 28 included in the inner bottom of each individual containing region is included in each individual containing region, and the identification code 27 is located at the center of the inner bottom of each individual containing region as much as possible, while paying attention to selection and orientation of the composite partition members 20, 20′, 50, and 60. The medicine and/or the medical instruments 8 to be contained in the individual containing regions thus formed may be used as a medicine set for operation.

Next, a dispensing operator such as a pharmacist or a nurse places the medicine and/or medical instrument 8 in each individual containing region of the partitioned tray 10 (refer to FIGS. 2B and 14) one by one. The dispensing operator should place the medicine and/or the medical instrument 8 while visually checking that either or both of the identification code 27 and the medicine or medical instrument information display section 28 in the individual containing region match either or both of the identification code and the medicine or medical instrument information display section for the medicine and/or the medical instrument 8. Consequently, the operation of placing the medicine and/or the medical instrument 8 may be readily and quickly performed. Thus, the tray 10 containing the medicines and/or the medical instruments 8 necessary as the medicine set for operation or the like, is speedily completed (refer to FIGS. 2B and 14).

Then, the completed tray 10 including the medicines and/or the medical instruments 8 is temporarily loaded into the cart 9 (refer to FIG. 3). Next, preparation of the subsequent tray 10 that has been partitioned and placement of medicines and/or the medical instruments 8 in the partitioned tray 10 are repeated, thereby completing the trays 10 including the medicines and/or the medical instruments one after another. Then, the completed trays 10 are also loaded into the cart 9 one after another. The trays 10 including the medicines and/or the medical instruments 8 whose locations of use are the same or close to one another should be brought together and transported together. When the trays 10 including the medicines and/or the medical instruments 8 are all prepared or when a time-out occurs, the cart 9 is moved to the location of use such as an operation room to use the medicines and/or the medical instruments 8. Then, all or a part of the medicines and/or the medical instruments 8 that are large in number and contained in the trays 10 are taken out at the location of use. The used tray 10 where only one or more of the medicines and/or the medical instruments 8 that have not been used are left is loaded into the cart 9 from which the used tray 10 was taken out, or a different cart 9. The cart 9 loaded with the used tray 10 is transported, and is then brought in to the medical dispensing location.

Then, in the used tray 10 that has returned to the medical dispensing location, the all or the part of the medicines and/or the medical instruments 8 used in the operation or the like were taken out and are not present. Then, the used tray 10 is manually placed in the shooting range of the checking unit (30, 40) on the desk in order to check the use status of the medicines and/or the medical instruments 8 immediately or later (refer to FIG. 12).

Then, the used tray 10 is shot by the imaging device 30 from above, and the image data 41 is captured by the detection device 40 (refer to FIG. 13). States of the individual containing regions defined by the composite partition members 20, 20′, 50, and 60 and states of the medicines and/or the medical instruments 8 placed in the respective containing regions are photographed in the image data 41. The identification code 27 appears in the individual containing region from which the medicine and/or the medical instrument 8 has been taken out. The identification code 27 is, however, hidden in the individual containing region where the medicine and/or the medical instrument 8 remains.

When the image data 41 is input, the identification code 27 is read by the reading program 43, based on the code data 42, and use of the medicine and/or the medical instrument 8 is detected by the determination program 44, based on a result of reading the identification code by the reading program 43. Specifically, identification candidate regions are cut out from the image data 41, and identification code reading is tried for each cut-out region. When the identification code 27 can be read, the medicine and/or the medical instrument 8 associated with the identification code 27 is determined to have been used. Then, the used quantity of the medicines and/or the medical instruments 8 is counted by the counting program 45 for each type of the medicines or for each tray, based on a result of determination by the determination program 44. Then, a result of the counting is output from the output device 46. In this manner, when the used tray 10 is manually placed in the shooting range, the used tray 10 is automatically shot, and one or more of the medicines and/or the medical instruments 8 that have been taken out from the tray 10 and have been used is grasped for each tray. The grasped result is used for inventory and ordering management as well. The tray 10, for which checking of the use status of the medicines and/or the medical instruments 8 was completed, is transferred from the desk to a different storage rack to be provided for reuse or alteration.

In the first embodiment described above, the surface fastener is adopted as a specific example of a low-cost and handy attachment member, and the fastener member 12 is attached to the upper surface of the intermediate layer member 11. The fastener member 12 may be, however, directly attached to the inner bottom surface of the tray 10. By doing so, the intermediate layer member 11 may be omitted.

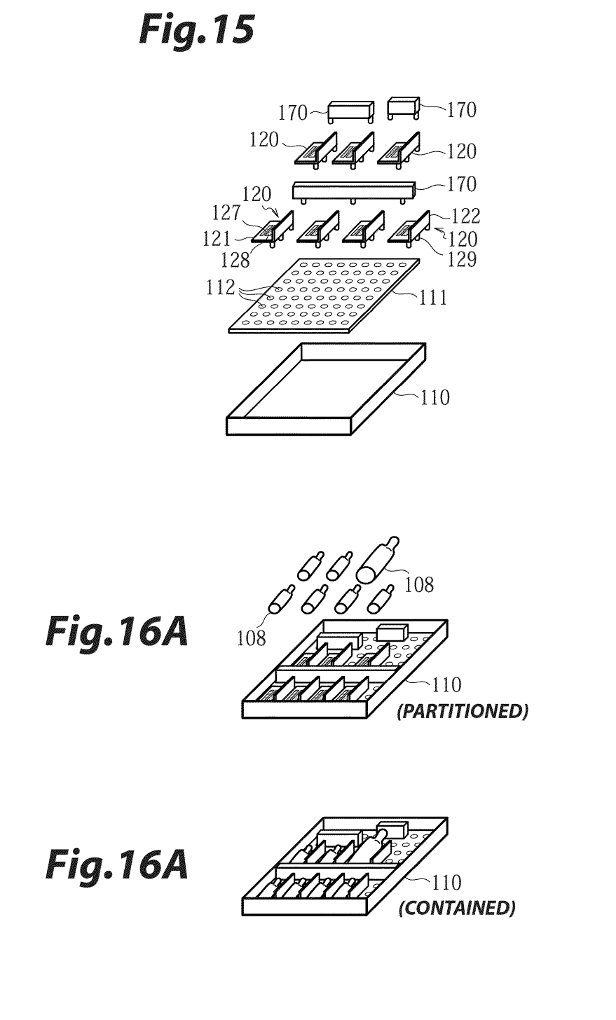

FIGS. 15 to 21 and FIG. 23 are diagrams used for explaining a second embodiment. Reference numerals obtained by adding 100 to the reference numerals of the components in FIGS. 1 to 14 are given to components that are the same as those in FIGS. 1 to 14 in the first embodiment. Description of these components will be thereby omitted. FIG. 15 is an exploded perspective view of a tray 110, an intermediate layer member 111, composite partition members 120, and partition members 170. FIG. 16A is a perspective view of the tray 110 in which individual containing regions are defined and medicines and/or medical instruments 108 to be contained in the tray 110. FIG. 16B is a perspective view of the tray 110 in which the medicine and/or the medical instruments 108 are contained.

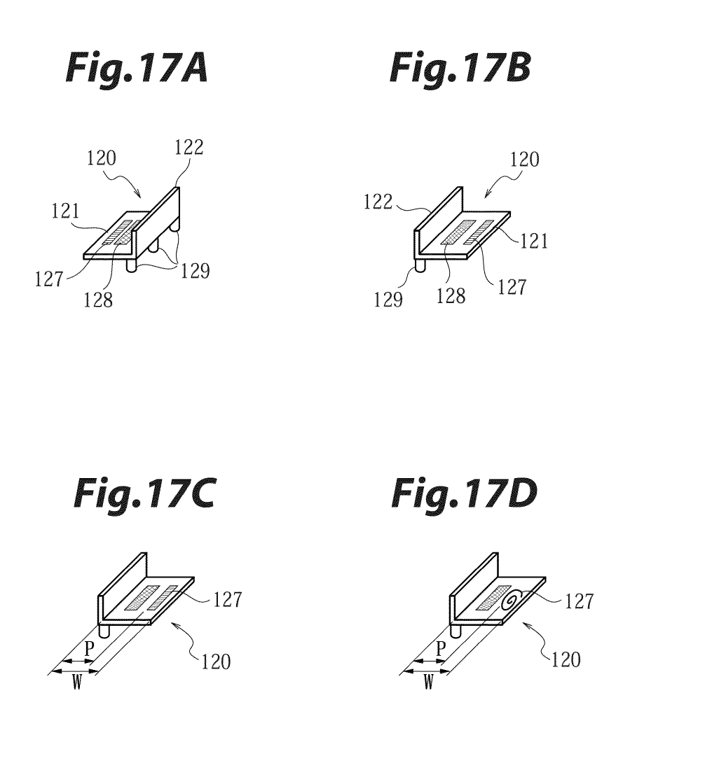

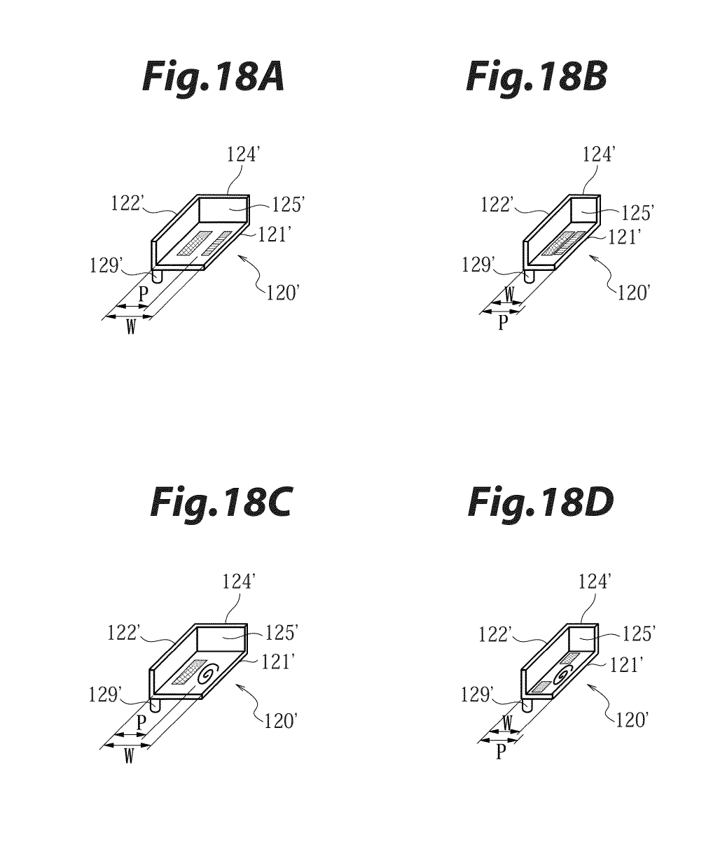

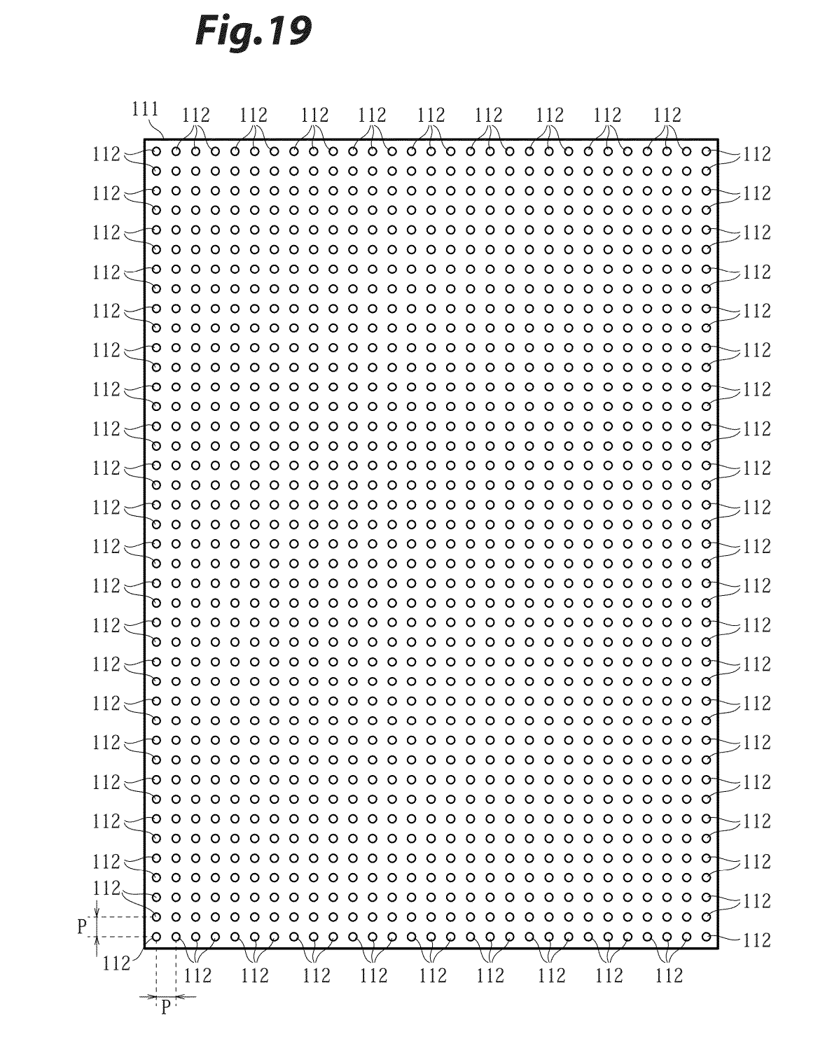

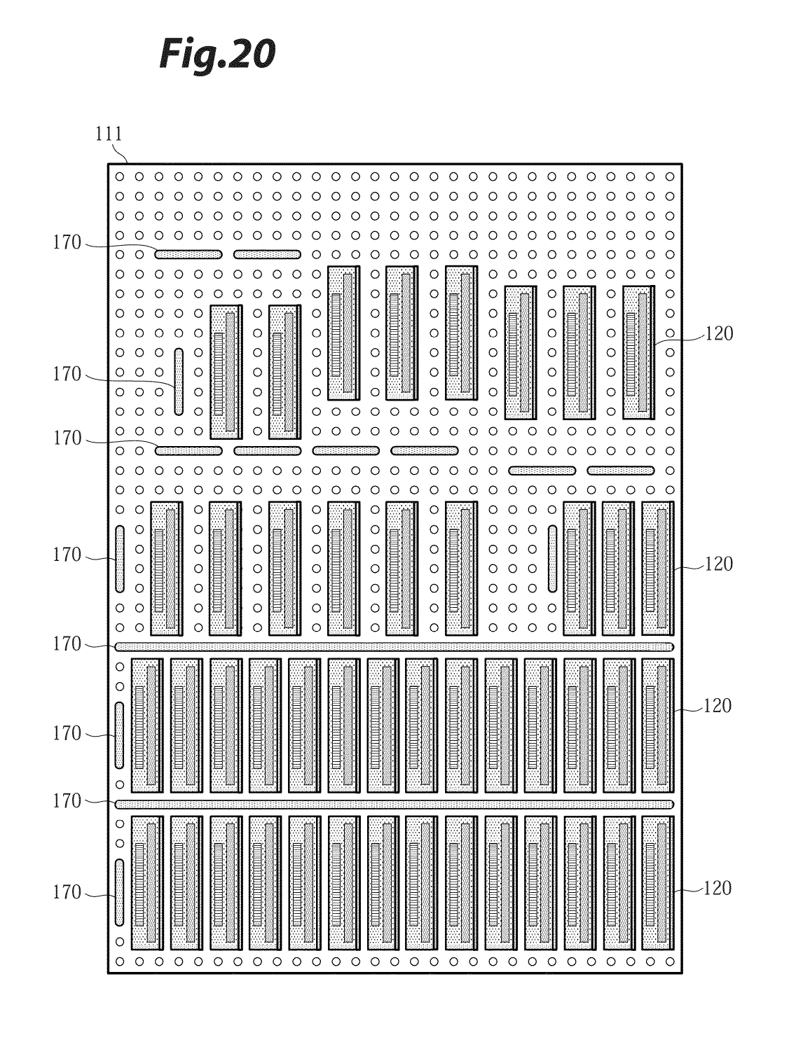

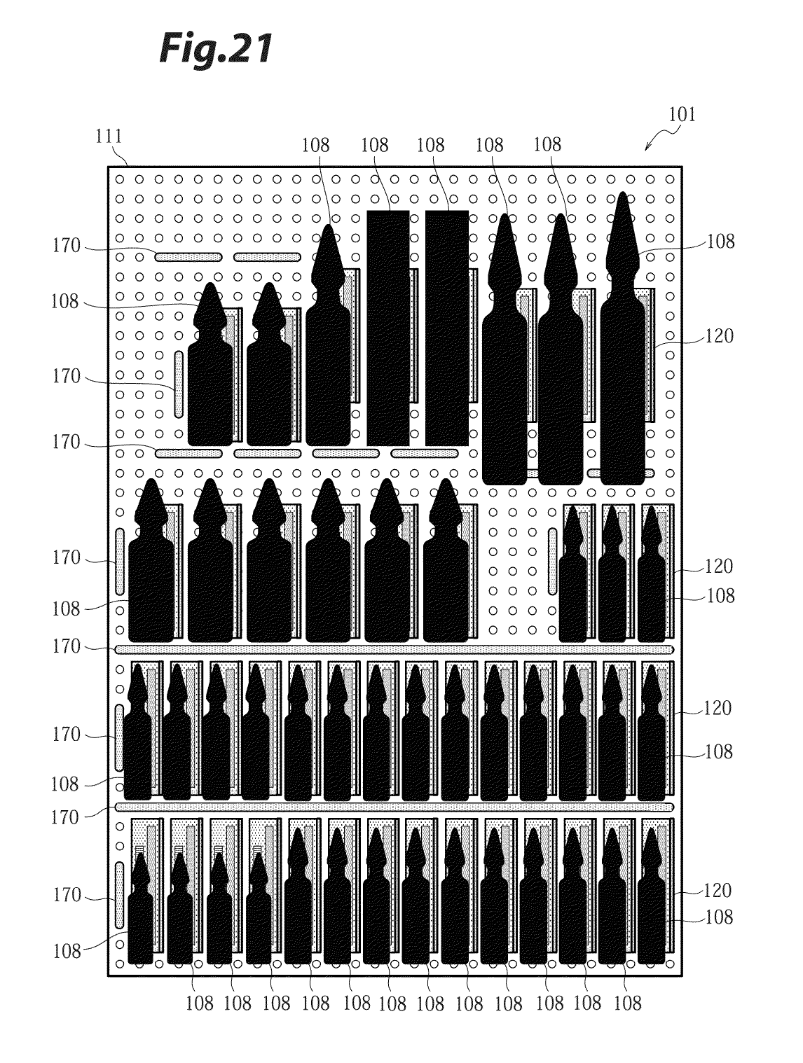

FIGS. 17A to 17D are each a perspective view of a composite partition member 120 including one partition portion. Further, FIGS. 18A to 18D are each a perspective view of a composite partition member 120′ including one partition portion 122′ and a connecting wall portion 124′. FIG. 19 is a plan view of the intermediate layer member 111, and FIG. 20 is a plan view of the intermediate layer member 111 in which the composite partition portions 120 and the partition members 170 are disposed, thereby defining a plurality of individual containing regions. FIG. 21 is a plan view of a management tray unit 101 in which the composite partition members 120 and the partition members 170 are disposed to define a plurality of individual containing regions and the medicines and/or medical instruments 108 are contained in the respective individual containing regions inside the tray 110.

In this embodiment, a columnar pin 129 is used as a positioning fitting portion, and a cylindrical hole 112 is used as a positioning fitted portion. A large number of the holes 112 are formed and arranged in the form of a matrix in the intermediate layer member 111 (refer to FIGS. 15 and 19). The matrix arrangement of the holes 112 is a horizontal and vertical two-dimensional arrangement as viewed in planar view. The large number of the holes 112 may have different horizontal and vertical pitches. When a large number of the holes 112 have the same horizontal and vertical pitches, the composite partition members 120 or the partition members 170 of same specifications may be used vertically and horizontally. Thus, the large number of the holes 112 in this embodiment have horizontal and vertical unit lengths of pitches P that are equal.

The composite partition members 120 and the partition members 170 are each a partition member to be disposed on the bottom wall portion of the tray 110 to partition an inside of the tray 110, thereby defining a plurality of individual containing regions. The composite partition member 120 and the partition member 170 are individually disposed on the bottom wall portion of the tray 110 by removably fitting the pins 129 into the holes 112 in the intermediate layer member 111 on an inner bottom of the tray 110 (refer to FIG. 18.

The composite partition members 120 each include a base portion 121 having a rectangular contour and a partition portion 122 extending along a first side of the base portion 121 and provided to coincide with the first side (refer to FIGS. 17A and 17B). A space 125 formed by the partition portion 122 constitutes one individual containing region by using another partition member or a side wall of the tray 110 (refer to FIGS. 20 and 21).

Similarly, the composite partition members 120′ in FIG. 18 each include a base portion 121′ having a rectangular contour, a partition portion 122′ extending along a first side of the base portion 121′ and provided to coincide with the first side, and a connecting wall portion 124′. A space 125′ formed by the partition portion 122′ and the connecting wall portion 124′ constitutes one individual containing region by using another partition member and a side wall of the tray 110.

The pins 129 and 129′ are each thin and cylindrical and projects downward. Thus, by inserting each of the pins 129 and 129′ into the hole 112, each of the composite partition members 120 and 120′ and the partition member 170 are removably fitted into the intermediate layer member 111.

While a medicine or medical instrument information display section 128 is attached to an upper surface section of the base portion 121 close to the partition portion 122, an identification code 127 is attached to an upper surface section of the base portion 121 distant from the partition portion 122. With respect to the composite partition member 120 having a width W of the base portion 121 longer than a unit length of pitch P (refer to FIGS. 17C and 17D) in particular, a whole or a part of the identification code 127 is attached to an upper surface section of the base portion 121 distant from the partition portion 122 by the unit length of pitch P or more. Machine reading of the whole or the part of the identification code 127 cannot be thereby performed when the whole or the part of the identification code 127 is hidden.

Assume that the identification code 127 is linear (refer to FIG. 17C). Then, even if a part of the identification code 127 along the whole length of the identification code 127 is exposed, machine reading of the part of the identification code 127 can be performed. Thus, it is safe to isolate the whole of the identification code 127 from the partition portion 122 by the unit length of pitch P or more. However, assume that the identification code 127 is not linear but spiral, for example (refer to FIGS. 17D, 18C, and 18D). Machine reading of the identification code 127 cannot be performed when the identification code 127 is hidden to an extent that apart of the spiral shape of the identification code 127 is disconnected. Thus, it is enough to isolate the part or more of the identification code 127 from the partition portion 122 or 122′ by the unit length of pitch P or more. With that arrangement, even when the medical and/or the medical instrument 108 is contained in the individual containing region having a width of three times or more of the unit length of pitch P with an allowance of the unit length of pitch P or less, the identification code 127 is certainly hidden by the medicine and/or the medical instrument 108 and cannot be read. Thus, accurate management of the medicine and/or the medical instrument 108 may be performed.

Assume, however, that the individual containing region has a width of twice the unit length of pitch P. Then, even when the identification code 127 is disposed in the individual containing region distant from the partition portion 122 or 122′ by the unit length of pitch P or more, the identification code 127 may be undesirably read if the medicine and/or the medical instrument 108 is close to the partition portion 122 or 122′. When the width W of each of the base portions 121 and 121′ is equal to or less than the unit length of pitch P (refer to FIGS. 18B and 18D), disposition of the identification code 127 is primarily impossible. In order to prevent occurrence of undesirable reading of the identification code 127 even in these cases, an upper surface of each of the base portions 121 and 121′ is inclined such that each of the base portions 121 and 121′ becomes thicker as it is more close to a corresponding one of the partition portions 122 and 122′ and becomes thinner as it is more distant from the corresponding one of the partition portions 122 and 122′ (as shown in FIG. 23). Then, the medicine and/or the medical instrument 108 will naturally move onto the identification code 127 to prevent the occurrence of undesirable reading of the identification code 127.

The partition members 170 each include a plate-like partition portion and a plurality of pins projectedly provided at lower end portions of the partition member 170 (refer to FIGS. 15, 20, and 21). Like each of the composite partition members 120 and 120′ mentioned above, the partition members 170 are each configured to be removably inserted into the intermediate layer member 111 by inserting the positioning fitting portions not shown into the holes 112.

In the above-mentioned second embodiment, the holes 112 are formed in the intermediate layer member 111. The holes 112, however, may be directly formed in an inner bottom portion of the tray 110. By doing so, the intermediate layer member 111 may be omitted.

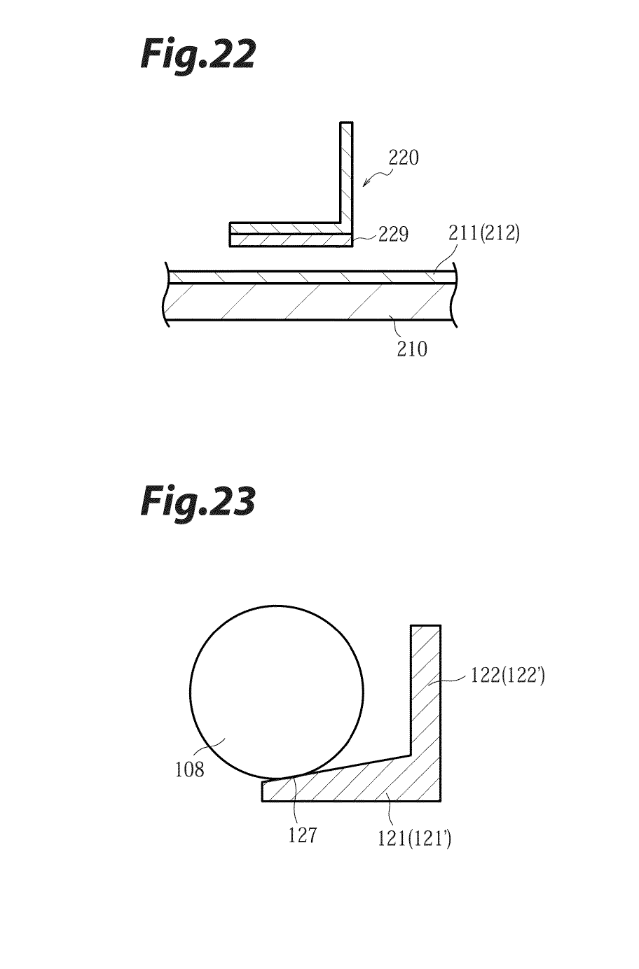

FIG. 22 is a diagram used for explaining a third embodiment. Reference numerals obtained by adding 200 to the reference numerals of the components in FIGS. 1 to 14 are given to components that are the same as those in FIGS. 1 to 14 in the first embodiment. Description of these components will be thereby omitted. In the third embodiment, a permanent magnet is used as a low-cost and handy attachment member. The bottom plate of a tray 210 or an intermediate layer member 211 may be set to the permanent magnet, and a permanent magnet 229 may be fixed to the bottom portion of a composite partition member 220, as shown in FIG. 22. The composite partition member 220 may be formed of the permanent magnet or a magnetic material. When the bottom plate of the tray 210 is formed of the permanent magnet, soft iron may be adopted for a whole or the base portion of the composite partition member 220. A magnetic attractive force is preferably strong in terms of stabilization for defining each individual containing region. On the other hand, the magnetic attractive force is preferably weak in order to allow the individual containing region to be readily defined again. For that reason, when the individual containing region is defined again, the tray 210 is placed on an electromagnet to temporarily weaken the magnetic attractive force. Then, the composite member 220 is thereby readily disposed again.

### Other Embodiments

In the above-mentioned embodiment, the used trays 10 are shot one after another. When the tray 10 is small and a plurality of the trays 10 can be arranged in the shooting range of the imaging device 30, the plurality of the trays 10 may be collectively shot at once.

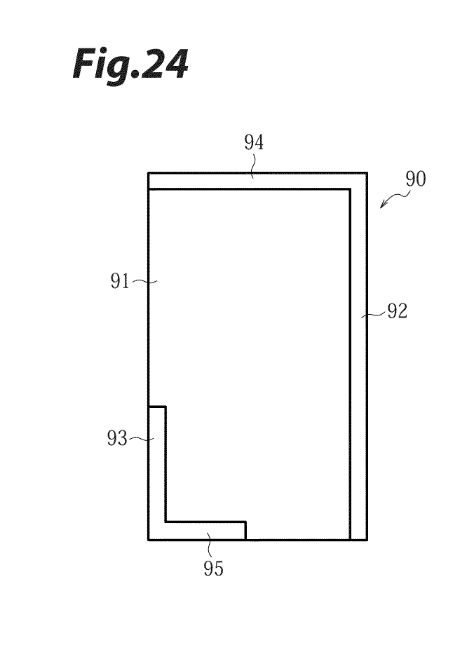

As a different specific example of a composite partition member, a partition portion or a connecting wall portion configured to coincide with a whole or a part of each of four sides of a base portion may also be provided. A composite partition member 90 shown in FIG. 24 includes a base portion 91 having a rectangular contour and a plurality of partition portions 92 and 93 extending along first and second sides of the base portion opposed to each other and arranged in parallel with a predetermined interval. The partition portion 92 adjacent to the first side is provided to coincide with the first side, and the partition portion 93 adjacent to the second side is provided to coincide with the second side. A connecting wall portion 94 connected to one end of the partition portion 92 and a connecting wall portion 95 connected to one end of the partition portion 93 are provided along third and fourth sides different from the first and second sides and opposed to each other.

In the above-mentioned embodiment, the checking unit (30, 40) is equipped together on the desk. The respective devices of the checking unit (30, 40) may be separately embodied. The checking unit may be mounted to a tray delivery cart as in the above-mentioned medicine management system using collective imaging of the inside of a tray (refer to Patent Document 2). Alternatively, the checking unit may be combined with the medicine management system.

In the above-mentioned embodiment, the detection device 40 is configured to determine that each medicine and/or the medical instrument 8 has been used immediately when the identification code 27 can be read from the image data 41. However, it may also so arranged that the detection device 40 even checks whether a result of determination by the detection device 40 about the use status of the medicine and/or the medical instrument 8 is checked by referring to the set data, like the determination program of the above-mentioned medicine management system using collective imaging of the inside of a tray (refer to Patent Document 2). To take an example, in a registration mode, a registration program configured to register a result obtained by the determination program 44 is additionally installed in the detection device 40. Then, before each medicine and/or the medical instrument 8 is contained in the partitioned tray 10, the partitioned tray 10 is subject to a process by the checking unit (30, 40), thereby registering a disposition state of each identification code 27 in the set data. Then, before the medicine and/or the medical instrument 8 is taken out from the tray 10 containing the medicine and/or the medical instrument 8 is used, the tray 10 containing the medicine and/or the medical instrument 8 is subject to a process by the checking unit (30, 40), thereby registering a disposition state of the identification code 27 in the set data. With that arrangement, a usage status of the medicine and/or the medical instrument 8 may be checked by checking a result of check of the used tray 10 against the set data.

### INDUSTRIAL APPLICABILITY

The management tray unit of the present invention may be used at various medical sites such as an operation room, an ICU, a hospital ward, and the like as well as the system for the in-house pharmacy as described above.

### REFERENCE SIGNS LIST

8 medicine and/or medical instrument
9 cart
10 tray
11 intermediate layer member
12, 29 surface fastener
20, 50, 60, 90, 120 composite partition member
21, 51, 61, 91, 121 base portion
22, 23, 52, 62, 63, 92, 93, 122, 123 partition portion
24, 54, 64, 94, 95 connecting wall portion
27 identification code
28 medicine or medical instrument information display section
30 imaging device (checking unit)
40 detection device (checking unit)
41 image data
42 code data
43 reading program
44 determination program
45 counting program
46 output device

1. A management tray unit comprising:
a tray configured to contain a medicine and/or a medical instrument;
a plurality of partition members disposed inside the tray, positioned with respect to a bottom wall portion of the tray, and configured to define a plurality of individual containing regions for individually containing the medicine and/or the medical instrument inside the tray;
the plurality of partition members including:
a plurality of composite partition members each configured to define one or more of the individual containing regions independently or in cooperation with another partition member, the plurality of composite partition members each including:
a base portion extending along the bottom wall portion and configured to be wholly or partially covered with the medicine or the medical instrument when the medicine or the medical instrument is contained in the individual containing region; and
one or more partition portions extending from the base portion in a direction away from the bottom wall portion;
an identification code provided on a surface of a section of the base portion of the composite partition member to be covered with the medicine or the medical instrument and configured to identify the medicine or the medical instrument contained in the individual containing region defined by the composite partition member, wherein the identification code is provided to be optically readable when the medicine or the medical instrument is not present on the section; and
an attachment member provided at each of the base portions of the composite partition members and the bottom wall portion of the tray, the attachment member forming an attachment configured to detachably attach the composite partition member to the bottom wall portion.

2. The management tray unit according to claim 1, wherein
the attachment is formed by a surface fastener comprising a fastener member including a hook surface and another fastener member including a loop surface, the attachment member provided at the bottom wall portion is one of the fastener member and the another fastener member of the surface fastener, and the attachment member provided at the base portion is the other of the fastener member and the another fastener member of the surface faster.

3. The management tray unit according to claim 1, wherein
the attachment has a structure configured to use a magnetic force of a permanent magnet.

4. The management tray unit according to claim 3, wherein
the attachment member provided at the bottom wall portion is one of the permanent magnet and a magnetic body to be attracted by the permanent magnet, and the attachment member provided at the base portion is the other of the permanent magnet and the magnetic body to be attracted by the permanent magnet.

5. The management tray unit according to claim 1, wherein
the attachment member provided at the bottom wall portion is formed of a plurality of positioning fitted portions arranged in a surface direction of the bottom wall portion at predetermined intervals, and the attachment member provided at the base portion is formed of one or more positioning fitting portions provided on a back surface of the base portion opposed to the bottom wall portion of the tray and configured to be fitted in one or more of the plurality of positioning fitted portions.

6. The management tray unit according to claim 5, wherein
the plurality of fitted portions are formed of a plurality of holes or concave portions formed in a plate disposed on the bottom wall portion in the form of a matrix, and the one or more fitting portions are formed of one or more convex portions configured to be fitted in the plurality of holes or concave portions.

7. The management tray unit according to claim 6, wherein
one or more of the plurality of composite partition members each include the base portion with a width longer than a unit length of pitch of the plurality of holes or concave portions, and a whole or part of the identification code is attached to an upper surface section of the base portion distant from the partition portion by the unit length or more, whereby machine reading of the whole or part of the identification code may not be performed when the whole or part of the identification code is hidden.

8. The management tray unit according to claim 1, wherein:
the plurality of composite partition members each include:
the base portion having a rectangular contour; and
a plurality of the partition portions extending along first and second sides of the base portion opposed to each other and arranged with predetermined intervals; and
a space formed between adjacent two of the plurality of the partition portions constitutes at least a section of the individual containing region.

9. The management tray unit according to claim 1, wherein:
the plurality of composite partition members each include:
the base portion having a rectangular contour; and
a plurality of the partition portions extending along first and second sides of the base portion opposed to each other and arranged with predetermined intervals;
a space formed between adjacent two of the plurality of the partition portions constitutes at least a section of the individual containing region;
the plurality of the partition portions are provided in parallel with the first and second sides; and
a connecting wall portion configured to connect one end of each of the plurality of the partition portions is provided at the base portion along one of third and fourth sides opposed to each other and different from the first and second sides.

10. The management tray unit according to claim 1, wherein:
the plurality of composite partition members each include:
the base portion having a rectangular contour; and
a plurality of the partition portions extending along first and second sides of the base portion opposed to each other and arranged with predetermined intervals;
a space formed between adjacent two of the plurality of the partition portions constitutes at least a section of the individual containing region;
the plurality of the partition portions are provided in parallel with the first and second sides;
a connecting wall portion configured to connect one end of each of the plurality of the partition portions is provided at the base portion along one of third and fourth sides opposed to each other and different from the first and second sides; and
each of the plurality of the partition portions adjacent to the first side is provided to coincide with the first side, and each of the plurality of the partition portions adjacent to the second side is provided apart from the second side.

11. The management tray unit according to claim 1, wherein:
the plurality of composite partition members each include:
the base portion having a rectangular contour; and
a plurality of the partition portions extending along first and second sides of the base portion opposed to each other and arranged with predetermined intervals;
a space formed between adjacent two of the plurality of the partition portions constitutes at least a section of the individual containing region;
the plurality of the partition portions are provided in parallel with the first and second sides;
a connecting wall portion configured to connect one end of each of the plurality of the partition portions is provided at the base portion along one of third and fourth sides opposed to each other and different from the first and second sides; and
each of the plurality of the partition portions adjacent to the first side is provided apart from the first side, and each of the plurality of the partition portions adjacent to the second side is provided apart from the second side.

12. The management tray unit according to claim 1, wherein:
the plurality of composite partition members each include:
the base portion having a rectangular contour;
one of the partition portions extending along one of first and second sides of the base portion opposed to each other; and
a connecting wall portion extending along one of third and fourth sides opposed to each other and different from the first and second sides and connected to one end of the partition portion.

13. The management tray unit according to claim 1, wherein:
the plurality of composite partition members each include:
the base portion having a rectangular contour;
one of the partition portions extending along one of first and second sides of the base portion opposed to each other; and
a connecting wall portion extending along one of third and fourth sides opposed to each other and different from the first and second sides and connected to one end of the partition portion;
the partition portion is provided at the base portion to coincide with the one of the first and second sides; and
the connecting wall portion is provided at the base portion to coincide with one of the third and fourth sides.

14. The management tray unit according to claim 1, wherein
the base portion becomes thinner as it is more distant from the partition portion, whereby an upper surface of the base portion is inclined.

15. The management tray unit according to claim 1, wherein
information capable of identifying the medicine or the medical instrument by mere visual observation is displayed on an upper surface of the base portion, in addition to the identification code.
